(19) United States
(12) Patent Application Publication
(10) Pub. No.: US 2002/0161388 A1
(43) Pub. Date: Oct. 31, 2002

(54) Elastomeric balloon support fabric

(76) Inventors: Sam L. Samuels, Landenberg, PA (US); Peter Popper, Wilmington, DE (US); William G. DiMaio, Boothwyn, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/084,545

(22) Filed: Feb. 25, 2002

Related U.S. Application Data
(60) Provisional application No. 60/271,770, filed on Feb. 27, 2001.

(51) Int. Cl. B32B 1/08; A61M 29/00; F16L 1/00; B65D 1/00; B29D 22/00

(52) U.S. Cl. 606/192; 428/36.9; 606/195

(57) ABSTRACT

Balloon catheter covers are elastic fabric structures of interconnected yarns, the structure having a high degree of stretch and recovery in the circumferential direction with little change in dimension in the longitudinal direction during multiple pressurization cycles over full range of inflation and deflation of the balloon catheter. The covers have longitudinal yarns positioned at about zero degrees to the balloon axis and reversibly-stretchable, circumferential yarns positioned at a high angle Ø to the axis.

A method for making the balloon catheter covers allowing nearly orthogonal placement of circumferential and longitudinal yarns involves triaxial braiding a minimum number of elastomeric braid yarns with multiple axial yarns to provide maximum convergence angle (approaching 90 degrees).

This application claims the benefit of U.S. Provisional Application No. 60/271,770, filed Feb. 27, 2001.

### BACKGROUND OF INVENTION

1. Field of Invention

The present invention relates to balloon catheters used in a variety of surgical procedures and particularly to elastomeric balloon support fabrics used to form elastomeric sleeves or balloon covers for use with balloon catheters. It also relates to a process for making such fabrics.

2. Background Discussion and Related Art

Balloon catheters of various forms are commonly employed in a number of surgical procedures. These devices comprise a thin catheter tube that can be guided through a body conduit of a patient such as a blood vessel and a distensible balloon located at the distal end of the catheter tube. Actuation of the balloon is accomplished through use of a fluid filled syringe or similar device that can inflate the balloon by filling it with fluid (e.g., water or saline solution) to a desired degree of expansion and then deflate the balloon by withdrawing the fluid back into the syringe.

In use, a physician will guide the balloon catheter into a desired position and then expand the balloon to accomplish the desired result (e.g., clear a blockage, or install or actuate some other device). Once the procedure is accomplished, the balloon is then deflated and withdrawn from the blood vessel.

There are two main forms of balloon catheter devices. Angioplasty catheters employ a balloon made of relatively strong but generally inelastic material (e.g., polyester) folded into a compact, small diameter cross section. These relatively stiff catheters are used to compact hard deposits in vessels. Due to the need for strength and stiffness, these devices are rated to high pressures, usually up to about 8 to 12 atmospheres depending on rated diameter. They tend to be self-limiting as to diameter in that they will normally distend up to the rated diameter and not distend appreciably beyond this diameter until rupture due to over-pressurization. While the inelastic material of the balloon is generally effective in compacting deposits, it tends to collapse unevenly upon deflation, leaving a flattened, wrinkled bag, substantially larger in cross section than the balloon was when it was originally installed. Because of their tendency to assume a flattened cross section upon inflation and subsequent deflation, their deflated maximum width tends to approximate a dimension corresponding to one-half of the rated diameter times pi (π). This enlarged, wrinkled bag may be difficult to remove, especially from small vessels. Further, because these balloons are made from inelastic materials, their time to complete deflation is inherently slower than elastic balloons.

By contrast, embolectomy catheters employ a soft, very elastic material (e.g., natural rubber latex) as the balloon. These catheters are employed to remove soft deposits, such as thrombus, where a soft and tacky material such as latex provides an effective extraction means. Latex and other highly elastic materials generally will expand continuously upon increased internal pressure until the material bursts. As a result, these catheters are generally rated by volume (e.g., 0.3 cc) in order to properly distend to a desired size. Although relatively weak, these catheters do have the advantage that they tend to readily return to their initial size and dimensions following inflation and subsequent deflation.

While balloon catheters are widely employed, currently available devices experience a number of shortcomings.

First, as has been noted, the strongest materials for balloon construction tend to be relatively inelastic. The flattening of catheter balloons made from inelastic materials that occurs upon inflation and subsequent deflation makes extraction and navigation of a deflated catheter somewhat difficult. Contrastingly, highly elastic materials tend to have excellent recovery upon deflation, but are not particularly strong when inflated nor are they self-limiting to a maximum rated diameter regardless of increasing pressure. This severely limits the amount of pressure that can be applied with these devices. It is also somewhat difficult to control the inflated diameter of these devices.

Second, in instances where the catheter is used to deliver some other device into the conduit, it is particularly important that a smooth separation of the device and the catheter occur without interfering with the placement of the device. Neither of the two catheter devices described above is ideal in these instances. A balloon that does not completely compact to its original size is prone to snag the device causing placement problems or even damage to the conduit or balloon. Similarly, the use of a balloon that is constructed of tacky material will likewise cause snagging problems and possible displacement of the device. Latex balloons are generally not used for device placement in that they are considered to have inadequate strength for such use.

Inventions described in U.S. Pat. Nos. 5,752,934; 5,868,704; and 6,120,477, all to Campbell et al. and all incorporated herein by reference, are intended to solve the limitations. The inventions disclosed in these patents, particularly the “balloon covers”, are taught as being useful for

1. creating a catheter balloon that is small and slippery for initial installation, strong for deployment, and returns to its compact size and dimensions for ease in removal and further navigation following deflation;

2. providing a catheter balloon that will remain close to its original compact pre-inflation size even after repeated cycles of inflation and deflation; and

3. strengthening elastic balloons, to provide them with distension limits and provide them with a lubricous outer surface.

The covers taught in the Campbell et al. patents are made of layers of PTFE film helically wrapped over other layers of PTFE film. On expansion, the angle of the wraps with respect to the axis of the balloon they cover decreases. To return to the pre-inflation diameter, it is necessary to apply tension to the balloon cover parallel to the longitudinal axis or to employ a cured elastomeric layer applied to the luminal surface of the cover to assist in recollapse.

Nevertheless, although the “balloon covers” taught in the Campbell et al. patents may have low profile and good trackability, and are able to expand and provide stress support to the balloon, they still leave various needs to be solved. In particular they appear to a) shrink longitudinally when expanding circumferentially and increase in length when contracting, b) require externally applied mechanical action (e.g., longitudinal extension) to recollapse or deflate the balloon, c) employ an elastomeric layer over the cover to assist in recollapse thereby increasing the bulkiness of the cover, d) restrict the flexibility of the balloon.

### SUMMARY OF INVENTION

The balloon covers of the present invention comprise an elastic fabric structure of interconnected yarn, the structure having a high degree of stretch and recovery in the circumferential direction. Preferably, the structure has little if any stretch in the longitudinal direction with the high degree of stretch and recovery in the circumferential direction. The longitudinal yarn preferably is not so elastic as the circumferential yarn and most preferably is a relatively inextensible yarn. By using a relatively inextensible longitudinal yarn and a reversibly-elastic circumferential yarn, the resulting covers are longitudinally stable (i.e. exhibit little or no dimensional change in the longitudinal direction upon expansion and collapsing in the circumferential direction) while being reversibly, and repeatedly expandable and collapsible in the circumferential direction. Preferably, the elastic yarns are selected so that the elastic sleeve (balloon cover) can achieve an expanded dimension of more than two times, even more than 2½ times, the collapsed dimension.

Preferably the longitudinal yarns of the cover are positioned at about zero degrees to the balloon axis, and the reversibly-elastic, circumferential yarns are positioned at a high angle Ø to the axis, preferably 70° or greater, particularly 85° or greater, and most preferably near 90° to the longitudinal yarns. By using elastic circumferential yarn, there is little if any change in circumferential yarn angle Ø in the expanded and unexpanded states.

Preferably, the fabric structure is a triaxial braided structure wherein the braiding yarn (circumferential yarn) is a reversibly-elastic yarn and the axial yarn is relatively inextensible.

By employing yarn to make the fabric structure, elastic sleeves, very low profile or thickness (less than about 0.25 millimeters) and very small diameters (less than 1.3 millimeter) can be achieved. Extremely small sizes (diameters) in both pre-inflation and deflated states, even after repeated inflations and deflations, are possible allowing for use of balloons inserted through small, tortuous paths in applications such as those involving the brain, liver or kidney in addition to cardiovascular applications.

The fabric of the present invention may be made by any known method (e.g., woven, knitted, braided, or bonded), but preferably is made by braiding, preferably on a circular braider. Preferably, the balloon covers are made of fabric that is braided by a new braiding process configuration that allows nearly orthogonal placement of the braiding and axial yarns. The new process configuration involves braiding with a minimum number of elastomeric braid yarns to provide maximum braiding angle (approaching 90 degrees). Preferably, very high angle Ø (with respect to axis) braid is achieved when using multiple axial yarns for stability (preferably more than 8) and relatively few braiding yarns (preferably fewer than 4). A preferred case employs 16 axials and 2 braiders. While it is possible to use a higher number of braiding yarns to achieve faster manufacturing, the braid angle Ø will become smaller as the number of braiding yarns increase.

The preferred fabric sleeve (balloon cover) is a tubular braid made of 16 axials interbraided by only 2 braiding yarns. The axial yarns are preferably relatively inextensible yarns (e.g. polyester) oriented parallel to the braid axis. The braiding yarns are preferable highly extensible yarns (e.g. spandex) oriented at an angle close to 90 degrees from the braid axis.

The braiding tension of the elastomeric yarns should be adjusted to accomplish two features: 1. when the balloon is collapsed, the elastomeric sleeve (balloon cover) should be under residual stress and impose a compacting pressure on the balloon; 2. when the balloon is inflated to its maximum desired diameter, the braiding yarns should be close to their maximum extension, at which time they will have substantially increased resistance to further extension. Under these conditions, the elastic fabric sleeve will minimize the size of the deflated balloon. Furthermore, the sleeve will provide the structure with a bicompliant response in which the balloon expands with a low modulus initially and a higher modulus as the balloon reaches the maximum desired diameter. This characteristic is particularly useful. It provides for ease of inflation, strength when inflated, and rapid, mechanically assisted deflation. It gives the surgeon an added degree of sensitivity in finally sizing the stent during deployment. Bicompliant characteristics can be given to otherwise monocompliant balloons.

The braiding yarns used in the present invention can be made of one or more monofilament and/or multifilament elastomeric yarns. Suitable elastomeric yarns can be made from spandex fibers or fibers of polyurethane polymers; silicone elastomers; polyester/polyether block copolymers, such as Hytrel® polyetherester available from E. I. du Pont de Nemours and Company; polypropylene; fluoroelastomers; elastomeric polyolefins; and suitable combinations thereof. Other suitable fibers include those fibers having a Young's modulus similar to the aforementioned elastomeric fibers. Preferably, the yarns are made from spandex fibers, preferably those in which the segmented polyurethane in the spandex fiber is selected from polyetherurethaneurea and/or polyesterurethaneurea block copolymers.

The elastic yarns can be covered with a hard yarn using any of a number of textile processes such as wrapping or jet entangling. The resulting yarn will process more effectively than a bare yarn and will provide a “hard stop” to limit extension. The negatives of using a covered elastic yarns are less total elongation and greater thickness of the resulting sleeve.

Longitudinal yarns used in the present invention can be made from fibers of polyesters, such as polyethylene-terephthalate (PET), including Dacron® available from E. I. du Pont de Nemours and Company; polyamides; aramids such as Kevlar® available from E. I. du Pont de Nemours and Company; polyolefins, such as polyethylenes and polypropylenes; polyglycolic acids; polylactic acids; fluoropolymers, such as polytetrafluoroethylene (PTFE; Teflon® available from E. I. du Pont de Nemours and Company); and suitable combinations thereof. Preferably, the fibers are polyester or, particularly if lubricity is important, PTFE.

The elastomeric sleeves or balloon covers of the present invention meet or exceed all the advantages of the prior art balloon covers and also a) remain dimensionally stable longitudinally while being inflated and deflated, b) rapidly and reversibly recollapse upon release of internal pressure without need of longitudinal tension or an added elastomeric layer over the cover, c) have a good balance of elasticity without added bulk, and d) do not significantly reduce flexibility of the balloon. It is particularly easy to engineer properties such as compliance or modulus and strength of the sleeve along its profile. The covers of this invention can be used for the same wide range of applications as set forth in the Campbell et al. patents.

Balloons covered by the sleeves of the present invention collapse rapidly (in less than 500 msec) and symmetrically to a low profile size (to nearly the initial pre-inflation size, particularly to a size that is less than 10% larger than the pre-inflation size) upon release of internal pressure. The cover provides force to expel fluid from the balloon to allow smooth, rapid and complete deflation to low profile. The rapid, symmetrical recollapse of the balloon after angioplasty or stent deployment allows for improved recross.

Being made of an fabric made of interconnected yarn (e.g., braided yarn), these sleeves can provide a “textured” surface that provides better retention and delivery of devices such as stents (preventing movement and allowing for more accurate positioning). These covers provide improved burst strength (shielding the balloon from membrane stresses), and, in the event of catastrophic balloon failure, contain the balloon fragments for easy retraction without surgical intervention. These covers virtually eliminate any tendency for the balloon to “pancake.” These covers over embolectomy balloons provide limits on inflation diameter and provide sufficient strength to allow use of embolectomy balloons for angioplasty applications and device placement. These elastic sleeves can support inflated balloon loads of greater than 200 pounds per square inch. Particularly when using a circular braider to make the sleeve, it is possible to provide increased strength of the cover at the distal and proximal ends of the balloon by varying the sleeve profile. This can be done by providing added braids positioned over the distal and proximal ends of the balloon. This configuration permits the balloon to inflate at its middle prior to inflating at its ends as is desired for stent placement.

Processes that can be used to fabricate sleeved balloon assembly include the following:

1. The elastic yarn may be braided over removable mandrel sized for the expanded balloon. The mandrel may be removed and the balloon inserted in a manner that the sleeve can contract around balloon.

2. The elastic yarn may be braided over removable mandrel overwrapped with removable coil sized for the expanded balloon. The mandrel may then be removed and the balloon inserted. The coil can then be removed to allow the sleeve to contract around balloon.

3. The elastic yarn may be braided over a removable mandrel sized for the deflated balloon followed by removal of mandrel and insertion of balloon. Braider tension may be adjusted to control expansion.

4. The elastic yarn may be braided over expanded balloon on catheter followed by allowing the resulting sleeve to contract and deflate the balloon to low profile.

5. The elastic yarn may be braided over a folded balloon on catheter with tension of braider yarns adjusted to control expansion.

Optionally, the elastic yarn may be woven with the inelastic yarn instead of being braided. See Example 2.

While it is preferable to make the structure that forms the balloon cover by making the fabric by interconnecting the yarns directly into a tubular form as discussed above, it is possible to make a flat fabric and then sew the edges together in manner that results in elastic yarns in the resulting tubular structure being in the circumferential direction.

### BRIEF DESCRIPTION OF THE DRAWINGS

FIGS. 1A, 1B and 1C depict, respectively, a balloon covered by elastic sleeve without pressure applied to the balloon, the balloon with high pressure applied, and the balloon after pressure is released. Figures show reversible circumferential expansion/contraction with essentially no change in longitudinal dimension (L).

FIGS. 2A and 2B show the microstructure of a triaxially braided sleeve with relatively inextensible axial yarn and interlaced elastic braiding yarn. Figure indicates high angle (Ø) of braid yarn to axial (longitudinal) yarn. FIG. 2B depicts the braided yarn without axials.

FIG. 3 shows a schematic view of a circular braider for braiding sleeve onto a tubular mandrel. The circular braider is equipped with multiple tubes through which axial yarn is fed and two carriers that move along serpentine path and through which elastic braiding yarn is fed.

FIG. 4A depicts a “spiral wire” form of a mandrel and FIG. 4B depicts a “water snake” form of a mandrel for use in place of tubular mandrel of FIG. 3.

FIG. 5 shows a schematic view of a circular braider for braiding sleeve directly onto inflated balloon catheter instead of the tubular mandrel as depicted in FIG. 3.

FIG. 6 shows a schematic view of a circular braider for braiding sleeve directly onto a deflated balloon catheter instead of the tubular mandrel as depicted in FIG. 3.

FIGS. 7A, 7B, and 7C depict a method of inserting a balloon into an expanded elastic sleeve supported on a tubular mandrel.

FIG. 7C shows the sleeve collapsed onto the balloon after removal of mandrel.

FIG. 8 shows balloon inserted in sleeve that is stretched over “spiral wire” form of mandrel. The mandrel is shown partially withdrawn, allowing collapse of sleeve onto balloon

FIGS. 9A and 9B depict the method of inserting a balloon into the “water snake” form of mandrel.

FIG. 9A shows the balloon at the start of insertion.

FIG. 9B shows the balloon almost fully inserted.

FIG. 10 is a plot of the diameter of an elastic-sleeve-covered balloon as a function of inflation pressure showing bicompliance achieved when using elastic sleeve of the present invention.

FIG. 11 is a plot showing inflation dynamics of same elastic-sleeve-covered balloon for which data in FIG. 10 was obtained. Diameter and inflation pressure are plotted as functions of time.

FIG. 12 is a plot showing deflation dynamics of the same elastic-sleeve-covered balloon for which data in FIG. 10 was obtained. Diameter and inflation pressure are plotted as functions of time.

### DETAILED DESCRIPTION OF INVENTION

Catheter Balloons

The catheter balloons employed in the present invention include any balloon catheter devices known in the art. In particular, the balloon catheters employed in the present invention may be angioplasty balloon catheters made of relatively strong but generally inelastic material such as polyester or embolectomy balloon catheters made of soft, very elastic material such as natural rubber latex.

Elastic Sleeves (Balloon Covers)

The balloon covers of the present invention are tubular comprising an elastic fabric structure of interconnected circumferential and longitudinal yarns as described herein. By interconnected, it is meant that the yarn or fibers are woven, weft or warp knitted, bonded, or braided, preferably triaxially braided. Preferably, the fabric structure is a triaxial braided structure wherein the braiding yarn (circumferential yarn) is a reversibly-elastic yarn and the axial yarn is relatively inextensible. The tubular form of the balloon cover can be made by braiding, weaving, weft or warp knitting, or bonding (making a non-woven fabric) the longitudinal and circumferential yarns directly into a tubular form. The tubular form can also be made by first making a flat fabric by braiding, weaving, weft or warp knitting, or bonding (making a non-woven fabric) longitudinal yarns and yarns that will be the circumferential yarns when made into a tubular form and then sewing two edges of the fabric running in the longitudinal direction together so as to form tubular structure.

The balloon covers of the present invention have a high degree of stretch and recovery in the circumferential direction and preferably little if any change in longitudinal dimension over the full range of circumferential change. Preferably, the balloon cover stretch in the circumferential direction is greater than two times, more preferably greater than 2½ times, still more preferably greater than 3½ times. Preferably, the balloon cover retains its elasticity during its service life and recovers a substantial amount of any imposed extension. Preferably, as the balloon cover's diameter is changed by a factor “X”, it's length will change less than 0.25*X, more preferably less than 0.1*X.

Preferably, the cover is comprised of multiple axial yarns (preferably 8 or more, more preferably 16 or more) positioned essentially parallel to the sleeve axis at about zero degrees to the balloon axis, and reversibly-elastic, circumferential yarns (preferably a small even number, preferably 2, 4, or 6) are positioned at a high angle Ø to the axis, preferably 70° or greater, particularly 85° or greater, and most preferably near 90° to the axial yarns. Decreasing the number of the axial yarns will reduce the strength and the geometric stability of the balloon. Too many axial yarns will crowd the braiding yarns especially during circumferential contraction. In that case, the braiding yarns might buckle above the fabric surface and greatly increase wall thickness. Employing a higher numbers of circumferential yarns will result in a lower braid angle Ø to the axis. This will reduce circumferential strength and increase axial contraction during inflation.

The covers of the present invention expand and contract primarily due to the elasticity of the circumferential yarns. Preferably, most if not all of the circumferential expansion/contraction is based on the stretch of the fiber and not due to change in circumferential yarn angle Ø in the expanded and unexpanded states. Preferably, there is essentially no angle Ø change over the full range of circumferential change.

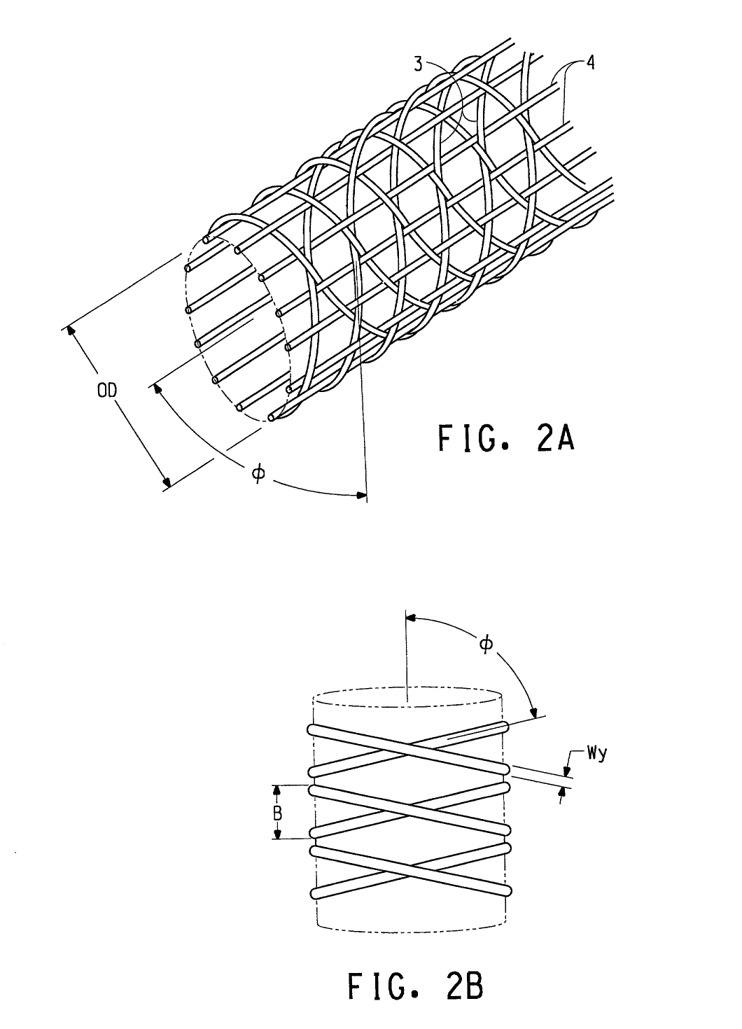

The Braiding Yarn Jamming Factor (defined as the ratio of braiding yarn width (Wy) to braiding yarn spacing (B) on FIG. 2B) can be used to define the desired constructions. Yarn spacing should be essentially the same for each wrap. Preferably, the Braiding Yarn Jamming Factor is: 1. less than approximately 0.8 to avoid braiding yarn overcrowding; and, 2. greater than 0.3 to insure mechanical stability. Preferably, the wall thickness of the elastomeric fabric sleeve is about 0.1 to 0.3 millimeters.

In another embodiment, the balloon cover sleeve may have added picks at locations that correspond to the proximal and distal ends of a stent deployment balloon to provide desirable “ends last” deployment of the stent (balloon and stent inflation first in middle and then moving to the ends).

In another embodiment, the balloon cover is shaped in a barrel or hour-glass shape. This is accomplished using convention braiding technology of braiding over a shaped mandrel.

In another embodiment, the balloon covers are bicompliant that is they have a higher compliance (preferably 0.02 to 0.06 mm/atm.) for moderate expansion and lower compliance (preferably less than 0.02 mm/atm.) when the covered balloon reaches near-maximum expansion. This characteristic is particularly useful. It provides for ease of inflation, strength when inflated, and rapid deflation when internal balloon pressure is released. The balloon covers of this invention provide bicompliant characteristics to otherwise compliant balloons.

Balloons covered by the balloon cover fabric of the present invention collapse rapidly (in less than 500 msec) and symmetrically to a low profile size (to nearly the initial pre-inflation size, particularly to a size that is less than 10% larger, preferably less than 5% larger than the pre-inflation size) upon release of internal pressure without need of longitudinal tension or an elastic membrane over-layer. The cover provides force to expel fluid from the balloon to allow smooth, rapid and complete deflation to low profile. The rapid, symmetrical recollapse of the balloon after angioplasty or stent deployment allows for improved recross.

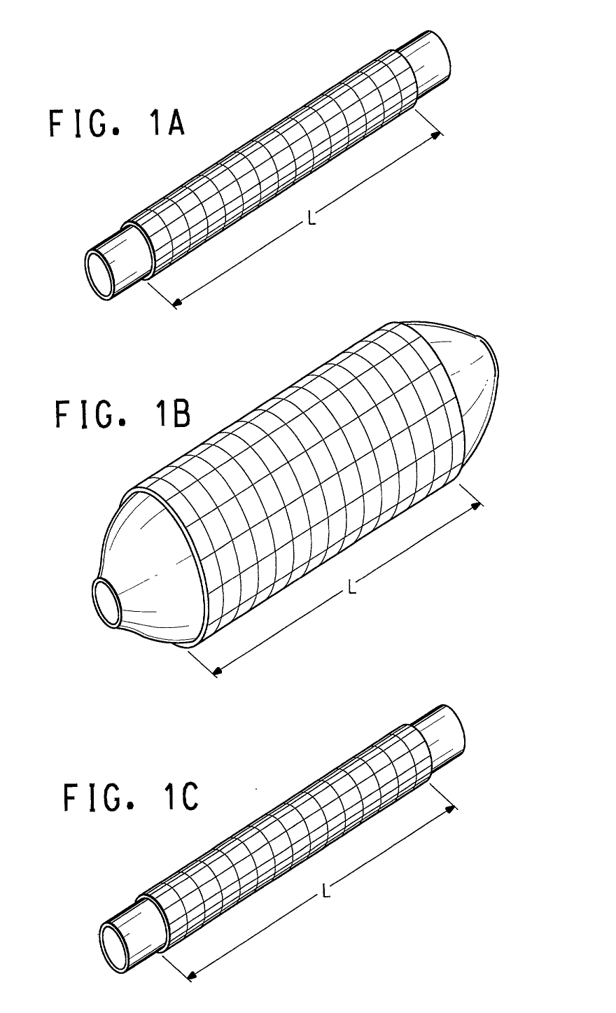

FIGS. 1A, 1B, and 1C depict three states of inflation of a balloon (2) inserted into the balloon cover or elastic sleeve (1) of the present invention. The balloon cover (1) is shown as having circumferential yarn (3) positioned at essentially 90° to the axial yarn (4). FIG. 1A shows the pre-inflated elastic sleeve covered balloon. FIG. 1B shows the inflated elastic sleeve covered balloon (inflated at least 2-3½ times or more) that results when high pressure is applied to the inside of the balloon. FIG. 1C shows the deflated elastic sleeve covered balloon in its contracted state (essentially the same diameter as the pre-inflated elastic sleeve covered balloon of FIG. 1A) which is rapidly reached following release of pressure from the inside of the balloon. In each of states depicted in the FIGS. 1A, 1B, and 1C, the longitudinal length, L, of the balloon cover (1) is essentially unchanged.

FIG. 2A shows a microstructure of a braided elastic sleeve/balloon cover of the present invention. Multiple axial yarns (4) run the longitudinal length of the sleeve. The axial yarn (4) is relatively non-compliant or is inextensible. Circumferential yarn (3) is triaxially braided with the axial yarn at a high braid angle Ø (not shown to scale) to form the sleeve fabric. The circumferential yarn (3), also referred to as braiding yarn, is a highly compliant, elastic yarn and is interlaced with the relatively non-compliant, inextensible axial yarn (4). FIG. 2B shows the effect of using two circumferential braiding yarns. The circumferential yarns cross two times during each braiding wrap. In FIG. 2B, the crossings for three wraps are depicted with only those on one side shown. The other cross in each wrap (not visible in the Figure) would be about 180° from the shown crosses.

Circumferential Yarn

Circumferential yarns (braiding yarns in a braided fabric) are selected so that the balloon cover fabric structure can stretch and recover in the circumferential direction. The circumferential yarns used in the present invention can be any elastomeric yarn capable of substantially recovering from large tensile deformation, preferably having an elongation to break of greater than 300% as measured according to ASTM (D13) Standard Tensile Tests. They preferably are selected from yarns that have the ability to stretch (deform) at least 250% under tension and then recover at least half of said deformation (preferably greater than 90 percent, preferably nearly 100% of the deformation) within one second after release of stretching tension.

The circumferential yarns used in the present invention can be made of one or more monofilament and/or multifilament elastomeric yarns. Suitable elastomeric yarns can be made from spandex fibers or fibers of polyurethane polymers; silicone elastomers; polyester/polyether block copolymers, such as Hytrel® polyetherester available from E. I. du Pont de Nemours and Company; polypropylene; fluoroelastomers; elastomeric polyolefins; and suitable combinations thereof. Other suitable fibers include those fibers having a Young's modulus similar to the aforementioned elastomeric fibers. Preferably, the yarns are made from spandex fibers, preferably those in which the segmented polyurethane in the spandex fiber is selected from polyetherurethaneurea and/or polyesterurethaneurea block copolymers.

The elastic yarns can be covered with a hard yarn using any of a number of textile processes such as wrapping or jet entangling. The resulting yarn will process more effectively than a bare yarn and will provide a “hard stop” to limit extension. The negatives of using a covered elastic yarns are less total elongation and greater sleeve thickness.

Preferably, these yarns have a denier of less than 100. Larger denier yarns can be used, but sleeve profile is sacrificed (resulting cover can become too thick and bulky) and openness of the resulting fabric becomes excessive. Lower denier yarns present manufacturing problems. The preferred denier can be chosen by one skilled in the art from the teachings herein so as to achieve the desired balance of properties.

The fabric must be strong enough to resist the internal pressure stresses ideally without assistance from the balloon material. For a thin walled cylinder, the maximum pressure stresses can be shown to equal pressure times max-radius (force/length) circumferentially and pressure times max-radius/2 (force/length) longitudinally. The fabric can be engineered to support these stresses by simply assuring that in each direction the yarn strength times the number of yarns per inch exceeds the imposed stress. That means that in each direction, for any given yarn, there will have to be at least a calculable number of yarns per inch.

In addition, each yarn selected will have a width that depends on its denier, density, and shape.

The combination of yarn width and required yarns per inch for may not be compatible with each other. To test this, a Jamming Factor was defined to be equal to Yarn Width(in) times Yarns/Inch. When this factor equals one, the yarns just touch; when it is greater than one they overlap; and when it is much less than one they have large gaps between them. Based on experience, the estimated acceptable range for the Jamming Factor is 0.3 to 0.8 for braiding yarns and 0.1 to 0.5 for axial yarns.

The design procedure for an acceptable fabric involves the following steps:

1. Establish the required internal pressure and maximum balloon diameter

2. Select the braiding and axial yarn types, properties, and deniers

3. Compute the yarn widths, required yarns per inch, and Jamming Factors

4. Iterate the selection of yarns to create a fabric with acceptable Jamming Factors and the minimum practical size yarn.

The following tables are useful for selecting the yarns and making the fabric of this invention. Table I identifies variables to be considered in the braiding and axial yarns. For any given yarn (braiding yarn or axial yarn), the yarn has a characteristic fiber strength, yarn weight/length, and fiber density. When used in a fabric, the characteristics associated with the yarn in the fabric include fabric strength efficiency, yarn packing, and yarn width/thickness. By choosing the yarns to be used and the desired yarn/fabric characteristics, then input values for the specific yarns from Table I can be used to calculate values in Table II using the equations in Table II (values are inserted for illustrative purposes). From iterative calculations, it is then possible to generate the data in Table III from which the appropriate yarn selection can be made.

TABLE I
Fabric Design Input Values

| | | | Variable |
|---|---|---|---|
| Variable | Units | Value | Name |
| Braiding Yarns (circumferential direction) | | | |
| Fiber strength | g/denier | .72 | gpd |
| Fabric str efficiency | | .9 | eff |
| Yarn wt/length | denier | 90 | denY |
| Fiber density | g/cc | 1.2 | rho |
| Yarn packing | | 1.0 | phi |
| Yarn width/thickness | | 1.0 | a |
| Yarn elongation (at max diameter) | % | 380* | e |
| Axial Yarns (longitudinal direction) | | | |
| Fiber strength | g/denier | 4.5 | gpd_w |
| Fabric str efficiency | | .9 | eff_w |
| Yarn wt/length | denier | 40 | denY_w |
| Fiber density | g/cc | 1.38 | rho_w |
| Yarn packing | | .9 | phi_w |
| Yarn width/thickness | | 3.0 | a_w |
| Imposed Loads/Geometry | | | |
| Pressure to inflate balloon | psi | 300* | p |
| Max sleeve diameter desired | mm | 3.8* | d |

*taken from Example 1

TABLE II
Fabric Design Calculated Values

| | | | Variable | |
|---|---|---|---|---|
| Variable | Units | Value | Name | Equation |
| Min sleeve | mm | 1.0 | dmin | =d*100/e |
| diameter | | | | |
| Stress-circum- | lb/in | 22.4 | Sh | =(p*d/2)/25.4 |
| ferential | | | | |
| Stress-long- | lb/in | 11.2 | Sa | =Sh/2 |
| itudinal | | | | |
| Strength/yarn- | lb | .128 | Syh | =gpd*denY*eff/454 |
| circumferential | | | | |
| Strength/yarn- | lb | .357 | Sya | =gpd_w*denY_{—} |
| longitudinal | | | | w*eff_w/454 |
| Min yarns/in- | 1/in | 175 | Yh | =Sh/Syh |
| circumferential | | | | |
| (at max dia) | | | | |
| Min yarns/in- | 1/in | 31 | Ya | =Sa/Sya |
| longitudinal (at | | | | |
| max dia) | | | | |
| Min no. axial | | 15 | Na | =Ya*d*pi()/25.4 |
| yarns in braid | | | | |
| Braiding Yarns | | | | |
| (circumferential | | | | |
| direction) | | | | |
| Yarn diameter | in | .0041 | Dys | =.000468*SQRT(denY/ |
| (equivalent solid | | | | rho) |
| rod) | | | | |
| Yarn diameter | in | .0041 | Dy | =Dys/SQRT(phi) |
| (equivalent | | | | |
| round) | | | | |
| Yarn thickness | in | .0041 | Ty | =Dy/SQRT(a) |
| Yarn width | in | .0041 | Wy | =SQRT(a)*Dy |
| Axial Yarns | | | | |
| (longitudinal | | | | |
| direction) | | | | |
| Yarn diameter | in | .0025 | Dys_w | .000468*SQRT(denY_{—} |
| (equivalent solid | | | | w/rho_w) |
| rod) | | | | |
| Yarn diameter | in | .0027 | Dy_w | =Dys_w/SQRT(phi_w) |
| (equivalent | | | | |
| round) | | | | |
| Yarn thickness | in | .0016 | Ty_w | =Dy_w/SQRT(a_w) |
| Yarn width | in | .0047 | Wy_w | =SQRT(a_w)*Dy_w |
| Fabric Geometry | | | | |
| Braid thickness | in | .0072 | Tbraid | =Ty + 2*Ty_w |
| before | | | | |
| expansion | | | | |
| Max braiding | 1/in | 247 | MaxY | =1/Wy |
| yarns/in | | | | |
| (side by side) | | | | |
| Max axial yarns/ | 1/in | 211 | MaxY_w | =1/Wy_w |
| in(side by side) | | | | |
| Jamming Factor- | | .71 | WpS | =Wy/(1/Yh) |
| braiding | | | | |
| yarns | | | | |
| Jamming Factor- | | .15 | WpS_w | =Wy_w/(1/Ya) |
| axial yarns | | | | |

By varying the yarns selected having the input variables of Table I and using the equations from Table II, the values in Table III can be generated for a range of deniers for an elastic braiding yarn with a given axial yarn. The values in Table III are for a spandex braiding yarn with a strength of 0.7 g/denier and density of 1.2 g/cc for a range of yarn deniers to be used with the selected 40 denier polyester axial yarn (4.5 grams/denier strength, minimum 31 yarns/inch at maximum diameter and minimum axial yarns in braid of 15) to make a sleeve that will support a pressure of 300 pounds per square inch and expand from 1 mm to 3.8 mm diameter. The values in Table I and II are for these yarns.

Note that each of these yarns could support the required pressure stresses, but with a differing number of yarns per inch. The lowest denier yarn to make a fabric without overlapping yarns is 50 denier. Although this yarn would make the thinnest fabric, it is more practical to use a heavier yarn, say 90 denier, to reduce the required number of yarns per inch.

TABLE III
Yarn Selection

| Braiding | | Braid Wall | Braiding Yarn | |
|---|---|---|---|---|
| Yarn Denier | Minimum | Thickness | Jamming | |
| (Wt./Length) | Yarns/In | (in.) | Factor | Comments |
| 10 | 1572 | .0045 | 2.12 | Not braidable |
| 20 | 786 | .0051 | 1.50 | Not braidable |
| 50 | 314 | .0062 | .95 | Borderline |
| 100 | 157 | .0074 | .67 | Acceptable |
| 200 | 79 | .0092 | .47 | Braid too open, |
| | | | | wall too thick |
| 500 | 31 | .0127 | .30 | Braid too open, |
| | | | | wall too thick |
| 90 | 175 | .0072 | .71 | Selected |
| | | | | construction |
| | | | | (Ex. 1) |

Longitudinal Yarn

Longitudinal yarns preferably are selected to resist stretching more than the circumferential yarns so that, when incorporated into the balloon cover, they restrict change in length of the balloon cover in the longitudinal direction over the full range of balloon expansion/contraction. Preferably, the longitudinal yarns have a secant modulus measured between zero stress and maximum axial stress (corresponding to maximum inflation pressure of the balloon) that is at least 5 times greater than the secant modulus of the circumferential yarns measured between zero stress and the maximum circumferential stress (corresponding to maximum inflation pressure of the balloon). The longitudinal yarns are relatively stiff (resist stretching), so that the balloon cover containing them is longitudinally stable. That is, the sleeve exhibits little or no dimensional change in the longitudinal direction over the full range of expansion and collapse in the circumferential direction.

Longitudinal yarns used in the present invention can be made from fibers of polyesters, such as polyethylene-terephthalate (PET), including Dacron® available from E. I. du Pont de Nemours and Company; polyamides; aramids such as Kevlar® available from E. I. du Pont de Nemours and Company; polyolefins, such as polyethylenes and polypropylenes; polyglycolic acids; polylactic acids; fluoropolymers, such as polytetrafluoroethylene (PTFE; Teflon® available from E. I. du Pont de Nemours and Company); and suitable combinations thereof. Preferable, the fibers are polyester.

The axial yarns can be selected by a procedure similar to that for the braiding yarns as more fully described above. The fabric must be strong enough to resist the internal pressure stresses ideally without assistance from the balloon material. For a thin walled cylinder, the maximum pressure stresses can be shown to equal pressure times max-radius (force/length) circumferentially and pressure times max-radius/2 (force/length) longitudinally. The fabric can be engineered to support these stresses by simply assuring that in each direction the yarn strength times the number of yarns per inch exceeds the imposed stress. That means that in each direction, for any given yarn, there will have to be at least a calculable number of yarns per inch. In addition, each yarn selected will have a width that depends on its denier, density, and shape.

For example, using the method described above for a balloon with a maximum diameter of 3.8 mm and a maximum pressure of 300 psi for polyester axial yarns with a strength of 4.5 g/denier and density of 1.38 g/cc, it can be shown that a 40 denier yarn gives an Jamming Factor in the acceptable range. This yarn requires at least 31 yarns per inch; and that corresponds to 15 total yarns.

For other size and pressure balloons, the required axial yarns might be different. The preferred fiber type is polyester in view of the large range of available products. Process for making Sleeve (and inserting balloon) The fabric of the present invention may be made by any known method (e.g., woven, knitted, braided, or bonded), but preferably is made by braiding, preferably on a circular braider. The tubular form of the balloon cover can be made by braiding, weaving, weft or warp knitting, or bonding (making a non-woven fabric) the longitudinal and circumferential yarns directly into a tubular form. The tubular form can also be made by first making a flat fabric by braiding, weaving, weft or warp knitting, or bonding (making a non-woven fabric) longitudinal yarns and yarns that will be the circumferential yarns when made into a tubular form and then sewing two edges of the fabric running in the longitudinal direction together so as to form tubular structure.

Preferably, the balloon covers are made of fabric that is braided by a new braiding process configuration that allows nearly orthogonal placement of the braiding circumferential yarns and axial yarns. The new process configuration involves braiding with a minimum number of elastomeric braid yarns to provide maximum braid angle (greater than 70°, approaching 90 degrees). Preferably, very high angle Ø (with respect to axis) braid is achieved when using multiple axial yarns for stability (preferably more than 8) and relatively few braiding yarns (preferably fewer than 4). In general, the number of braiders should be significantly less than the number of axials preferably by a factor of eight. This contrast sharply with conventional braiding in which there are typically twice as many braiders as axials. A preferred case employs 16 axials and 2 braiders. While it is possible to use a higher number of braiding yarns to achieve faster manufacturing, the braid angle Ø will become smaller as the number of braiding yarns increase.

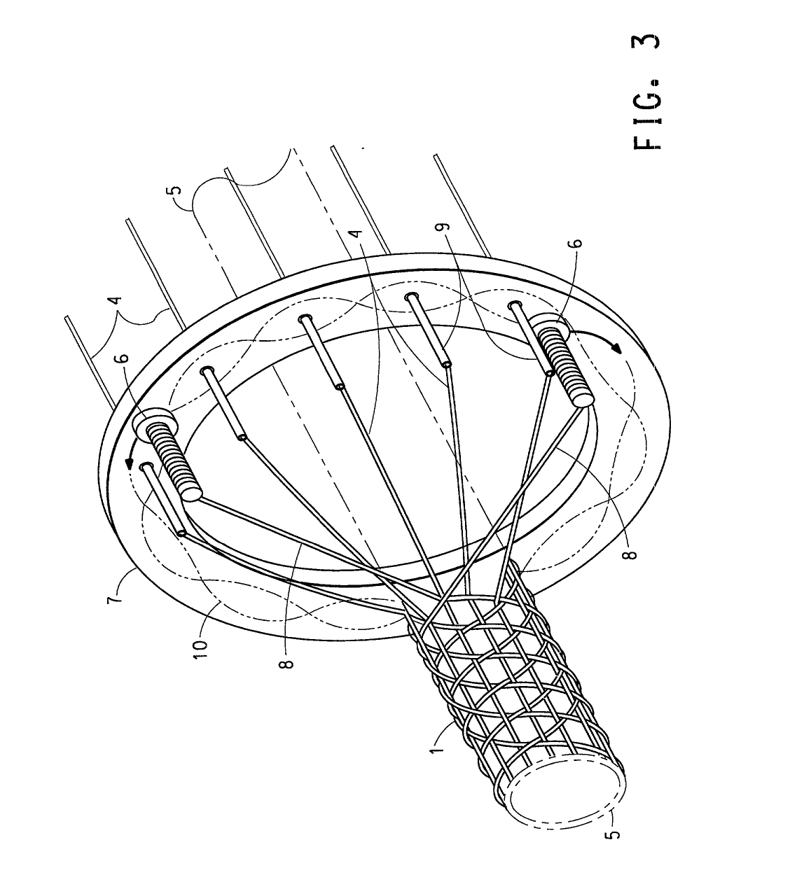

FIG. 3 depicting a circular braider can be used to explain the new braiding process. A tubular mandrel (5) is shown extending through (and centered in) the opening in the circular braiding plate (7) with a partially braided sleeve (1) on the mandrel. Low elongation axial yarns (4) are fed through multiple axial tubes (9) and laid down along the length of the mandrel (5) at essentially a zero degree angle to the mandrel and to the axial (longitudinal) direction of the sleeve (1) as the mandrel is advanced through the braider. As the mandrel is advanced through the braider, high elongation braiding yarns (8) from a small number (two in the process depicted) of braid carriers (6) are interlaced onto the mandrel. The braid carriers (6) move in opposite directions along a serpentine carrier path (10) positioned in the braiding plate (7) so as to cause the braiding yarn (8) to interlace with the axial yarns (4) and each other at the points where the braid carriers (6) cross paths. The mandrel (5) is advanced through the braiding machine at a rate adjusted to the speed of the braid carrier (6) movement along the serpentine carrier path (10) to assure desired cover. The rate of mandrel advance with respect to the revolutions/minute of the braid carriers should be adjusted so that the required number of braiding yarns per inch are deposited.

It should be noted that the mandrel can take various forms. FIG. 3 depicts a mandrel that has a diameter that is about the diameter of an expanded balloon catheter. When such a “large diameter” mandrel is used, the elastic braiding yarn (8) is laid down under the tension. The tension should be adjusted to be approximately the tension that the circumferential yarn will be under when the elastic sleeve covered balloon is in its expanded state. Tension is adjusted so that the yarn is stretched as it is interlaced with the axial yarns (4). Tension is controlled by adjusting the springs on the carriers. If the tension is too great, then the maximum balloon diameter will be restricted and braiding may be difficult. If the tension is too low, then the sleeve may not contract snugly over the folded balloon. Preferred tension when the mandrel is the “large diameter” size is approximately 15 g for a 90 denier spandex braiding yarn.

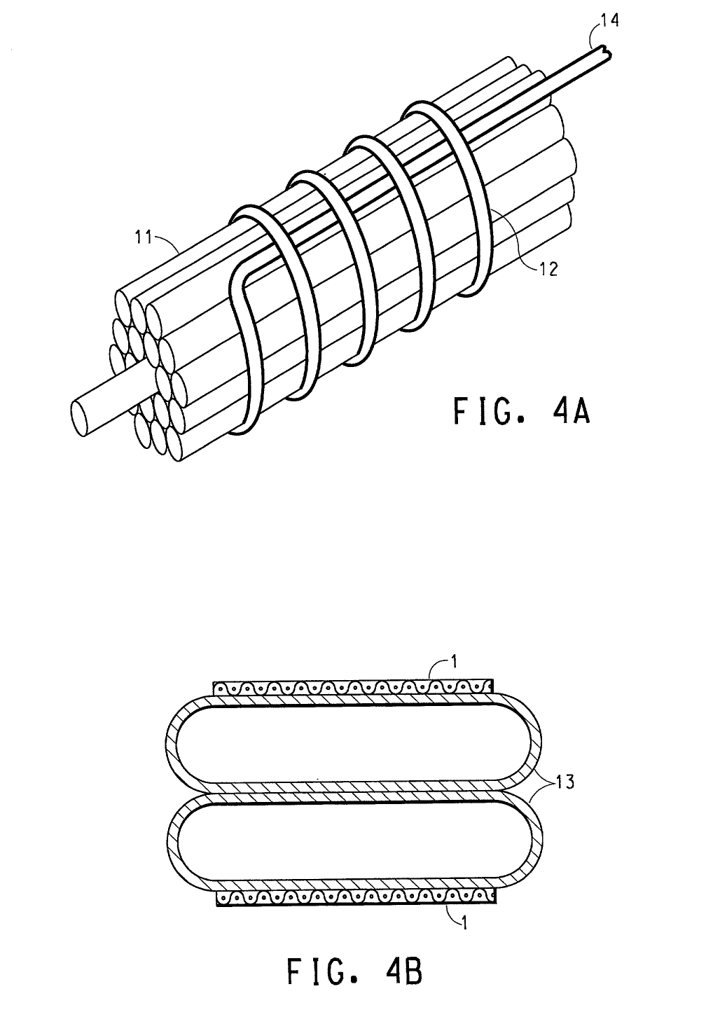

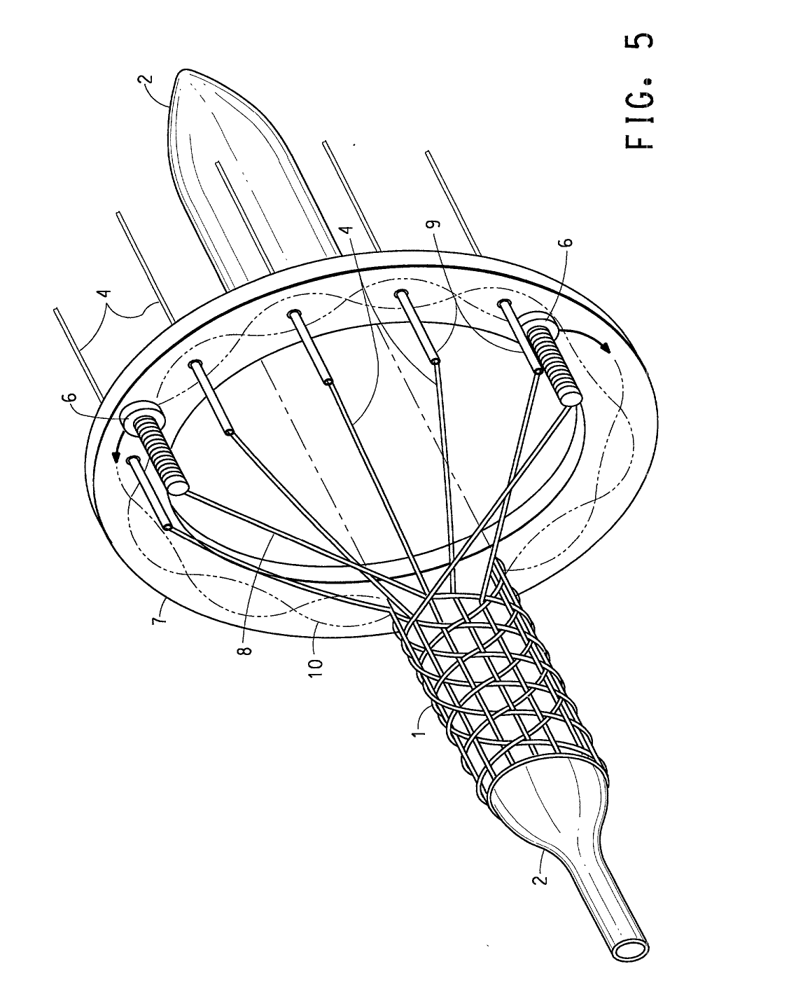

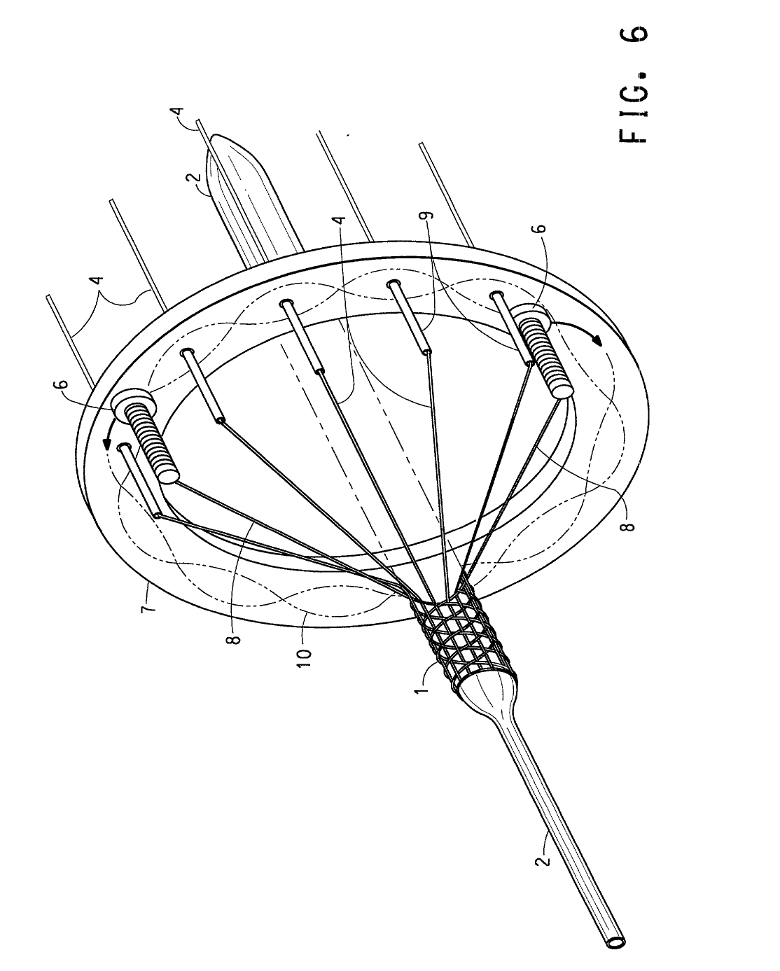

It should be noted that the mandrel can take various forms. FIG. 3 shows the mandrel as a tube. Examples of other forms are depicted in FIG. 4A, FIG. 4B, FIG. 5 and FIG. 6. The actual form the mandrel takes is not important so long as the balloon can be inserted into the completed sleeve.

It should be noted that the mandrel need not be cylindrical. A noncylindrical sleeve can be shaped as needed by braiding over a shaped mandrel.

It should be noted that the braiding yarn spacing, and consequently the resulting fabric modulus, can be profiled along the length of the catheter cover by varying the rate of braid formation relative to the machine rotation rate.

FIG. 4A shows a “spiral wire” or “coiled” mandrel. Wire (12) is lightly wound around a bundle of monofilaments (11) to provide structure to the mandrel. One end (14) of the wire (12) is preferably laid along the length of the bundle of monofilaments (11) to a point where the wire is bent so as to start winding circumferentially around the bundle (11) and back over the wire toward its starting end as shown in FIG. 4A.

FIG. 4B shows an elastic sleeve (1) over a pressurized torus (“water snake”) mandrel (13) that can be used in place of the tube mandrel of FIG. 3. The “water snake” is formed of two pressurized bladders in the shape of elongated torus with a minimal size hole.

FIG. 5 shows the same circular braider configuration as the one shown in FIG. 3, with the exception that the mandrel is an inflated balloon catheter (2). The braider is operated in the same manner as described with respect to the one in FIG. 3. As was the case with respect to the “large diameter” mandrel in FIG. 3, the tension of the braiding yarn (8) must be adjusted to the tension desired for the inflated balloon.

FIG. 6 shows the same circular braider configuration as the one shown in FIG. 3 and FIG. 5, with the exception that the mandrel is a deflated or folded balloon catheter (2). The braider is operated in the same manner as described with respect to FIG. 3. When the mandrel is the deflated or folded balloon (2), however, the tension of the braiding yarn (8) must be low enough that the braiding yarn (8) is interlaced with the axial yarns (4) in a relaxed state so that when the balloon in the sleeve is subsequently inflated, the tension is that desired for the inflated balloon.

Using the parameters in Tables I and II, the braiding process for the yarn selected using Table III can be operated according to Table IV.

TABLE IV
Braiding Machine Setup

| | | | Variable |
|---|---|---|---|
| | Units | Value | Name |
| Input Variables | | | |
| No. Carriers in Braiding Machine | | 32 | Nc |
| No. Carriers Used (No. Braiding | | 2 | Nb |
| Yarns) | | | |
| Braiding Yarn Width | in | .0041 | Wy |
| Braid angle (yarn to axis) | deg | 85 | Theta |
| Machine rotation rate | rpm | 5 | Mr |
| Jamming factor | | .71 | WpS |
| Calculated Value | | | |
| Braid take-off rate | in/min | .06 | Vb* |

*Vb = Nb*(Wy/WpS)*Mr/sin(Theta*pi)/180

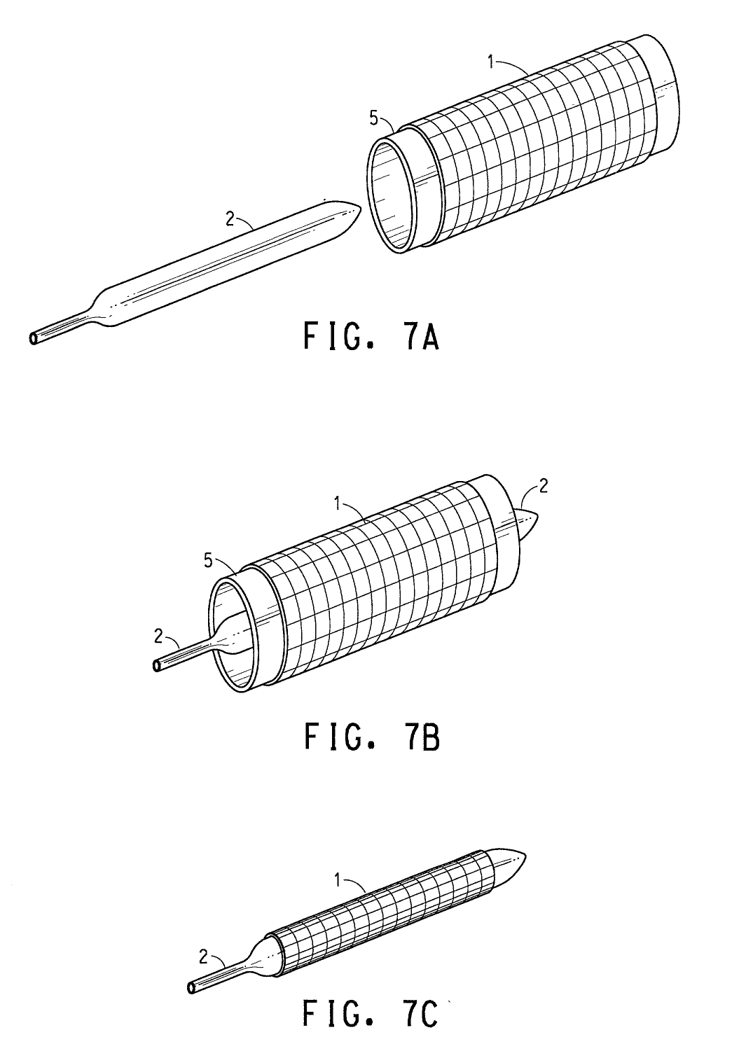

FIG. 7 shows one method of inserting the balloon into the elastic sleeve. In this case, FIG. 7A shows the elastic sleeve (1) is over a tubular, removable mandrel (5) with the deflated or folded balloon (2) attached to a catheter positioned for insertion into the tube. FIG. 7B shows the balloon (2) inserted into the tubular-mandrel-supported sleeve. The tubular mandrel (5) may, for example, be made of segments (not shown) that can be withdrawn once the balloon is in place, allowing the elastic sleeve (1) to contract (relieving the tension under which the sleeve braided) onto the balloon (2) as depicted in FIG. 7C.

FIG. 8 shows another method of inserting a deflated or folded balloon (2) into the elastic sleeve (1). In this case, the elastic sleeve (1) is stretched (under tension) over a coil of support wire (12) that can be formed as shown in FIG. 4. The deflated or folded balloon (2) is inserted into the area left when the monofilaments (see FIG. 4) are removed after the coil of the support wire (12) is formed. With the balloon (2) inserted, the end (14) of the wire running beneath the coiled portion of the support wire (12) toward the proximal end of the balloon, the elastic sleeve (1) will, starting at the distal end of the balloon, collapse onto the balloon.

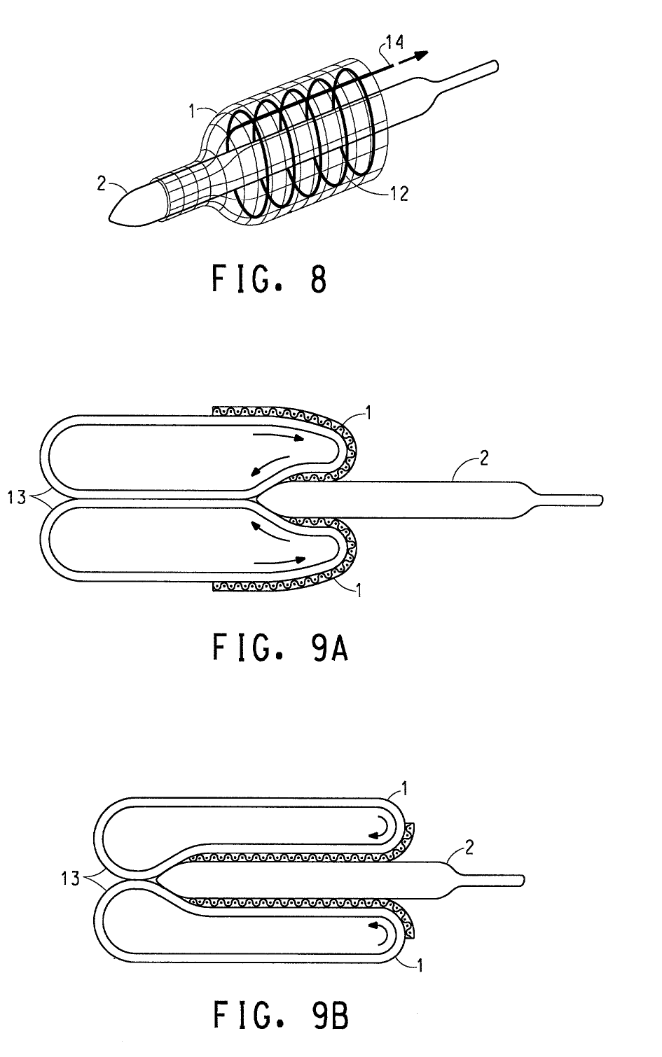

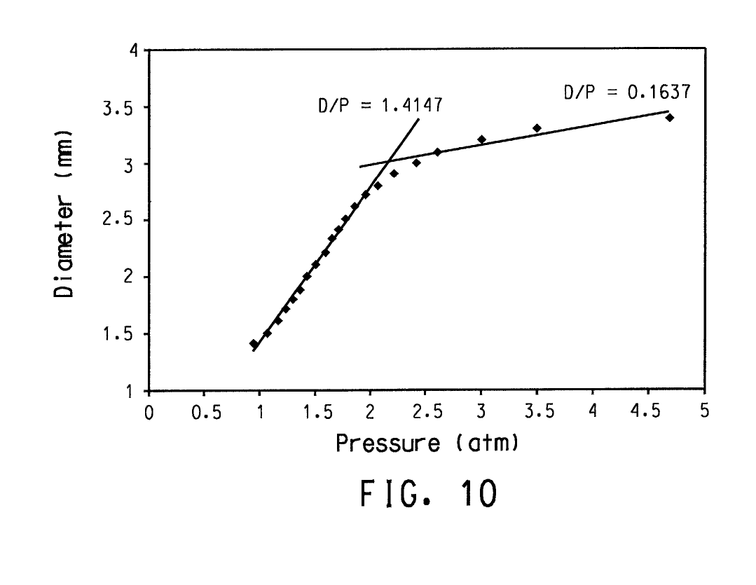

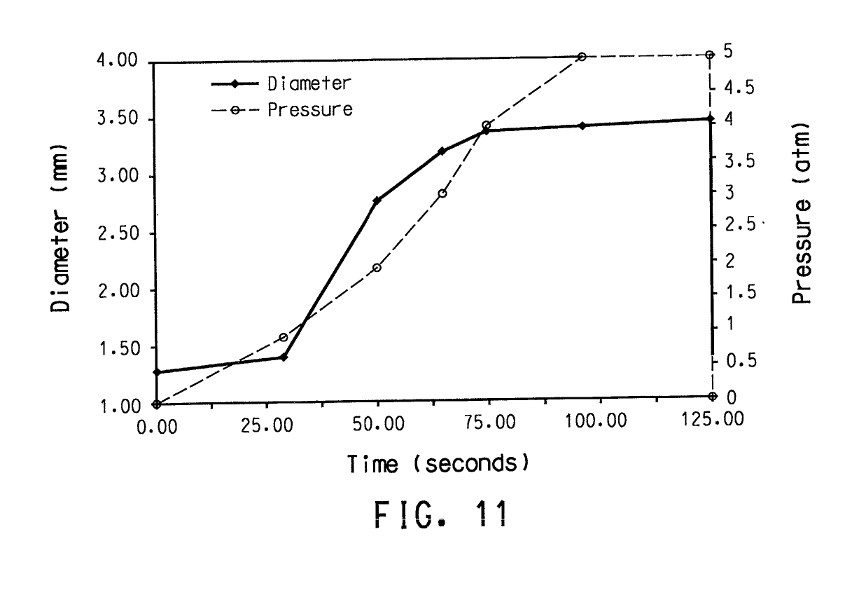

FIGS. 9A and 9B show still another method of inserting a deflated or folded balloon (2) into the elastic sleeve (1). In this case, the elastic sleeve (1) is under tension in its expanded state over the pressurized torus (“water snake”) mandrel (13). As the balloon (2) is inserted into the center of the “water snake” mandrel (13) as shown in FIG. 9A. As the balloon (2) is advanced through the “water snake” mandrel (13) as shown in FIG. 9B, the membrane forming the “water snake” will roll over on itself, carrying the elastic sleeve (1) with it so that it contracts onto the balloon (2) as the balloon (2) fully inverts the “water snake” (13).

The following examples describe in detail the construction of various embodiments of the balloon cover and catheter balloon of the present invention. Evaluation of these balloons is also described in comparison to conventional angioplasty and embolectomy balloons.

### EXAMPLES

### Example 1

### Braided Elastomeric Fabric Sleeve

Fabric Description

The yarns in this fabric of this example are interlaced in a tubular braided geometry. Sixteen axial yarns are oriented in the longitudinal direction, and they are interlaced by two braiding yarns. The braiding yarns lie in opposing helices that are nearly perpendicular to the longitudinal axis. There are approximate 254 braiding yarns per inch of tube length. The braid diameter can be varied from about 1 to 4 mm, depending on the internal pressure, with the length of the braid remaining essentially constant.

Yarn Materials

The axial yarns are made of polyester yarns (40 denier, 27 filaments). These yarns are generally inextensible with a break elongation of 27%. The braiding yarns, on the other hand, are made of spandex fibers with a break elongation of 600%.

The spandex yarns (90 denier) have a high degree of recovery from any imposed strain. The spandex yarns permit the braided tube to change diameter substantially. In the collapsed state the braid diameter is 1 mm and this grows to 3.8 mm in the expanded state.

Fabrication Method

The tube is braided on a conventional circular braider (New England Butt with 32 carriers and 16 axial positions). The machine is run with only 2 carriers, which carry the braiding yarns and run in opposing directions, and a full set of 16 axials. The braiding yarns are spandex and the axial yarns are polyester as described above.

To establish the size of the expanded state, the braid is formed over a removable mandrel that corresponds to the maximum diameter. A mandrel made of multiple monofilaments was used to facilitate removal after braiding. The mandrel was made of a cylindrical array of 14 polypropylene monofilaments, each with a diameter of 0.030 inches. This mandrel was removed, several monofilaments at a time, after braiding.

The braiding yarns were processed under moderate tension (approximately 15 grams). This provided a residual stress to the braid formed over the mandrel. When the mandrel was removed, the yarns simply retracted to a shorter length and the braid diameter decreased from 3.8 to 1 mm.

To achieve the 254 spandex yarns per inch, the takeoff rate was set relative to the rotations rate of the machine to approximately 0.13 inches per minute. The running speed was set to 5 rpm.

The wall thickness of the braid was approximately 0.2 mm.

### Example 2

### Woven Elastomeric Fabric Sleeve

Fabric Description

The yarns in this fabric are interlaced in a tubular woven geometry. Sixty ends (longitudinal yarns) are oriented in the warp direction and they are interlaced by the perpendicular filling yarn. There are approximately 90 picks (filling yarns) per inch of tube length. The tube diameter varies from about 1.3 to 4.5 mm, depending on the internal pressure, and the length of the tube remains essentially constant.

Yarn Materials

The longitudinal ends are made of polyester yarns (40 denier, 27 filaments). These yarns are generally inextensible with a break elongation of 27%. The filling yarn, on the other hand, is made of spandex fibers with a break elongation of 600%.

The spandex yarns have a high degree of recovery from any imposed strain. The spandex yarns permit the woven tube to change diameter substantially. In the collapsed state the woven diameter is 1.3 mm and this grows to approximately 4.5 mm in the expanded state.

Fabrication Method

The tube is woven on a captive shuttle tape loom using 60 warp yarns. Filling yarns are inserted at 90 picks/inch. The filling yarns are spandex and the warp yarns are polyester as described above.

To provide a convenient form for subsequent handling, the tube is woven over a removable mandrel. The mandrel consists of 120 polypropylene monofilaments, each with a diameter of 0.2 mm, which are woven into the tube through a single heddle on a separate harness. The monofilaments self-organize into a cylindrical mandrel in the core of the resulting woven tube. This mandrel can be easily removed, several monofilaments at a time, after weaving. When the mandrel was removed, the filling yarns retracted to a shorter length and the tube diameter decreased from about 2 mm to about 1.3 mm. Upon subsequent lateral stretching, the tube diameter reversibly increased to approximately 4.5 mm with no significant change in length.

The wall thickness of the woven tube was approximately 0.2 mm.

### Example 3

### Process for Braiding Elastomeric Fabric Sleeve Directly onto an Inflated Balloon Catheter

Fabric Description

The yarns in this fabric are interlaced in a tubular braided geometry. Sixteen axial yarns are oriented in the longitudinal direction and they are interlaced by two braiding yarns. The braiding yarns lie in opposing direction helices that are nearly perpendicular to the longitudinal axis. There are approximate 254 braiding yarns per inch of tube length. The braid diameter varies from about 1 to 4 mm, depending on the internal pressure, and the length of the braid remains essentially constant.

Yarn Materials

The axial yarns are made of polyester fibers (40 denier, 27 filaments). These yarns are generally inextensible with a break elongation of 27%. The braiding yarns, on the other hand, are made of spandex fibers with a break elongation of 600%.

The spandex yarns have a high degree of recovery from any imposed strain. The spandex yarns permit the braided tube to change diameter substantially. In the collapsed state the braid diameter is 1.3 mm and this grows to 3.5 mm in the expanded state.

Fabrication Method

The tube is braided on a conventional circular braider (New England Butt with 32 carriers and 16 axial positions). The machine is run with only 2 carriers, which carry the braiding yarns and run in opposing directions, and a full set of 16 axials. The braiding yarns are spandex and the axial yarns are polyester as described above.

An inflated balloon catheter (3.5 mm diameter, 5 atmospheres pressure). was fed through the core of the braiding machine just as the mandrel in example 1. The catheter had a non compliant polymeric balloon that could be pressurized with a manual pump (AVE Corp Model 9C03E14). The catheter used was an AVE Model 9C03E14 fitted with a 3.5 mm dia×16 mm long balloon.

The braiding yarns were processed under moderate tension (approximately 15 g) over the inflated catheter. This provided a residual stress to the braid formed over the inflated balloon. When the pressure was released, the yarns retracted to a shorter length and the balloon collapsed from its initial 3.5 mm to 1.3 mm.

Test Results

Self Folding

It is important to note that when the pressure was released, the sleeve forced the balloon to collapse and “self fold” into a small uniform cylinder. On subsequent inflation, the balloon expanded freely. This suggests that an elastic oversleeve of this invention can be used to fold a balloon and avoid the currently used balloon folding process.

Cyclic Loading

The sleeved balloon catheter was repeatedly inflated and deflated between 0 and 75 psi. Throughout these cycles the sleeve stays fixed on the balloon without any shifting.

Bicompliance

The mechanical performance of the sleeve was tested by inflating the balloon to varying pressures. The outside diameter was measured at each pressure. The results are plotted on FIG. 10. This graph clearly shows that the sleeved balloon initially expands readily (large diameter increase with increase of pressure). That diameter increase is primarily due to the balloon unfolding. At a particular diameter, the system stiffens and only a small increase in diameter occurs as the pressure is increases. This “bicompliant” behavior is considered desirable.

Inflation Dynamics

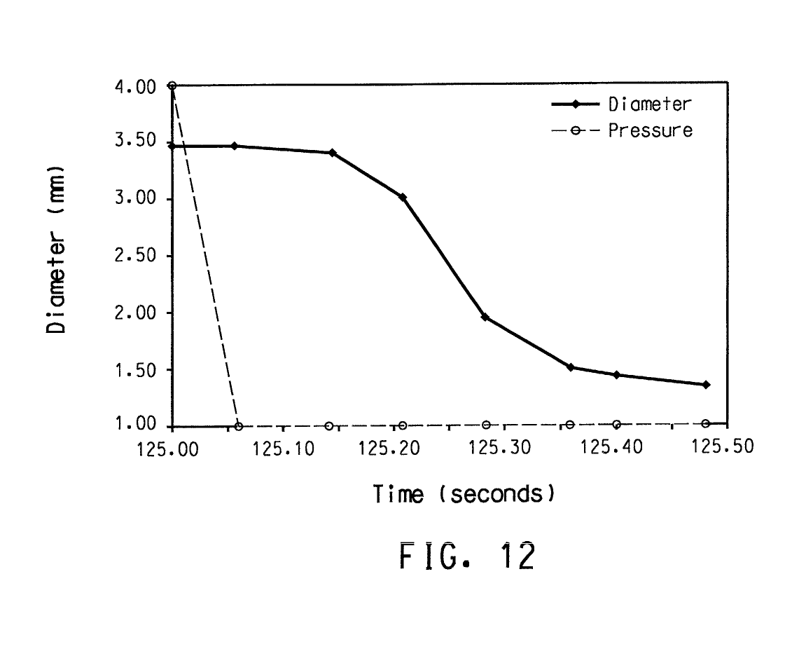

The data plotted on FIG. 10 can be shown in terms of its time sequence. FIG. 11 shows the pressure-time function of the imposed pressurization along with the diameter-time function measured.

Deflation Dynamics

The rapid collapse of the sleeved balloon is shown on FIG. 12. This graph shows that when the pressure is released the time to collapse completely is less than approximately 0.4 sec. Note that there is a time lag between pressure release and diameter collapse due to the fact that air, rather than saline was used in this test.

1. A balloon catheter cover comprising a tubular elastic fabric structure of interconnected circumferential and longitudinal yarns, the structure having a high degree of stretch and recovery in the circumferential direction.

2. The balloon catheter cover of claim 1 wherein there is essentially no change in length in the longitudinal direction over the full range of stretch and recovery in the circumferential direction.

3. The balloon catheter cover of claim 2 wherein the circumferential yarns have an elongation at break of more than 300%.

4. The balloon catheter cover of claim 3 wherein the longitudinal yarns have an elongation at break of less than 30%.

5. The balloon catheter cover of claim 1 wherein the degree of stretch in the circumferential direction is more than two times.

6. The balloon catheter cover of claim 1 wherein the degree of stretch is more than 3 times.

7. The balloon catheter cover of claim 1 wherein the longitudinal yarns are positioned at about zero degrees to the balloon axis and the circumferential yarns are positioned at an angle Ø to the axis of at least 70°.

8. The balloon catheter cover of claim 7 wherein the angle Ø is greater than 85°.

9. The balloon catheter cover of claim 8 wherein the angle Ø about 90°.

10. The balloon catheter cover of claim 1 wherein the fabric structure is a triaxial braid wherein the circumferential yarns are elastomeric braiding yarns and the longitudinal yarns are relatively stiff axial yarns.

11. The balloon catheter cover of claim 1 wherein the fabric structure is a woven fabric wherein the circumferential yarns are filling yarns and the longitudinal yarns are warp yarns.

12. The balloon catheter cover of claim 1 wherein the tubular structure is made from a fabric selected from the group of fabrics consisting of non-woven fabrics and those made by weft knitting and by warp knitting.

13. The balloon catheter cover of claim 12 wherein the tubular structure is made by sewing edges of a flat fabric together so as to make a tube having a longitudinal dimension and a circumferential dimension, the edges being sewn together being along the longitudinal dimension.

14. The balloon catheter cover of claim 1 wherein the elastomeric yarns are selected from the group consisting of yarns made from spandex fibers, fibers of polyurethane polymers, fibers of silicone elastomers, fibers of polyester/polyether block copolymers, fibers of polypropylene, fibers of fluoroelastomers, fibers of elastomeric polyolefins, and fibers of combinations thereof.

15. The balloon catheter cover of claim 14 wherein the elastomeric yarns are spandex fibers wherein the segmented polyurethanes of the spandex fibers is selected from the group consisting of polyetherurethaneurea and polyesterurethaneurea block copolymers, or combinations thereof.

16. The balloon catheter cover of claim 15 wherein the spandex fibers are covered with a hard yarn.

17. The balloon catheter cover of claims 1 wherein the longitudinal yarns are selected from yarns made from fibers of polyesters; polyamides; aramids; polyolefins; polyglycolic acids; polylactic acids; fluoropolymers; and combinations thereof.

18. A process for making a tubular structure having a longitudinal dimension and a circumferential dimension to be used as a balloon catheter cover in which multiple longitudinal yarns are placed from a fixed source along the longitudinal dimension over a core so as to not intertwine with themselves and multiple circumferential yarns from moving sources are intertwined in the circumferential dimension with the longitudinal yarns and themselves.

19. The process of claim 18 in which the core is a balloon catheter.

20. The process of claim 18 in which the core is a removable mandrel.

21. The process of claim 20 in which the removable mandrel is an array on monofilament yarns.

22. The process of claims 18 where the circumferential yarns are intertwined with the longitudinal yarns and themselves by flat or circular weaving.

23. The process of claims 18 where the circumferential yarns are intertwined with the longitudinal yarns and themselves by triaxial braiding.

24. The process of claims 18 where the circumferential yarns are intertwined with the longitudinal yarns and themselves by knitting.

25. A braiding process for making balloon catheter covers comprising triaxially braiding 2, 4 or 6 elastomeric braiding yarns with multiple axial yarns.

26. A triaxial braiding process wherein the number of braiding yarn is less than half of the number of axial yarns.

27. The triaxial braiding process of claim 26 wherein the number of braiding yarns is less than or equal to one-eighth of the number of axial yarns.

28. The braiding process of claims 25 wherein the braid angle between the braiding yarn and the axial yarn is greater than 70°.

29. A method of making a balloon catheter cover in which the balloon catheter cover is formed directly over a balloon catheter.

30. A method of making a balloon catheter cover in which the balloon catheter cover is formed over a removable mandrel and the cover is subsequently placed over a catheter balloon.

31. The method of claim 30 in which the removable mandrel is a helical spring which is removed by unwinding the coils of the spring from the inside to cause the catheter cover to contract onto a balloon catheter.

32. The balloon catheter cover of claim 1 in which the properties vary along the length of the sleeve

33. A balloon catheter cover of claim 32 in which the properties vary by changes of the yarn spacing

34. A balloon catheter cover of claim 1 in which the shape is not cylindrical.

35. A method of making a balloon catheter cover of claim 33 in which the non-cylindrical shape is obtained by the using of a shaped mandrel.
